(19) United States
(12) Patent Application Publication
(10) Pub. No.: US 2022/0339610 A1
(43) Pub. Date: Oct. 27, 2022

(54) CATALYST COMPRISING COKE AND PROCESS FOR THE PRODUCTION OF DIENES

(71) Applicants: VERSALIS S.P.A., San Donato Milanese (IT); ALMA MATER STUDIORUM - UNIVERSITA' DI BOLOGNA, Bologna (IT)

(72) Inventors: Juliana VELASQUEZ OCHOA, Bologna (IT); Fabrizio CAVANI, Modena (IT); Irene TOSI, Bologna (IT); Erika FARCI, Bologna (IT); Nicola VECCHINI, Mantova (IT)

(21) Appl. No.: 17/639,136

(22) PCT Filed: Aug. 25, 2020
(86) PCT No.: PCT/IB2020/057922

§ 371 Date: Feb. 28, 2022

(30) Foreign Application Priority Data
Aug. 27, 2019 (IT) 102019000015069

(51) Int. Cl. B01J 27/20; C07C 1/24; B01J 21/12; B01J 37/08

(52) U.S. Cl. CPC B01J 27/20; C07C 1/24; B01J 21/12; B01J 37/08

(57) ABSTRACT

A catalyst having coke wherein the coke, upon analysis by infrared spectroscopy in diffuse reflection, has at least two peaks at a wavelength between 1450 cm⁻¹ and 1700 cm⁻¹.

The aforesaid catalyst having coke can be advantageously used in a process for the production of a diene, preferably a conjugated diene, more preferably 1,3-butadiene, said process having the dehydration of at least one alkenol having a number of carbon atoms greater than or equal to 4.

Preferably, the alkenol having a number of carbon atoms greater than or equal to 4 can be obtained directly from biosynthetic processes, or through catalytic dehydration processes of at least one diol.

When the alkenol is a butenol, the diol is preferably a butanediol, more preferably 1,3-butanediol, even more preferably bio-1,3-butanediol, i.e. 1,3-butanediol deriving from biosynthetic processes.

When the diol is 1,3-butanediol, or bio-1,3-butanediol, the diene obtained with the process is, respectively, 1,3-butadiene, or bio-1,3-butadiene.

### TECHNICAL FIELD

The present disclosure relates to a catalyst comprising coke.

More in particular, the present disclosure relates to a catalyst comprising coke characterized in that said coke, upon analysis by infrared spectroscopy in diffuse reflection (“Diffuse Reflectance Infrared Fourier Transform Spectroscopy”—DRIFTS), has at least two peaks at a wavelength comprised between 1450 cm⁻¹ and 1700 cm⁻¹.

The aforesaid catalyst comprising coke can be advantageously used in a process for the production of a diene, preferably a conjugated diene, more preferably 1,3-butadiene, comprising the dehydration of at least one alkenol having a number of carbon atoms greater than or equal to 4.

Therefore, the present disclosure also relates to a process for the production of a diene, preferably a conjugated diene, more preferably 1,3-butadiene, comprising the dehydration of at least one alkenol having a number of carbon atoms greater than or equal to 4, in the presence of said catalyst comprising coke.

Preferably, said alkenol having a number of carbon atoms greater than or equal to 4 can be obtained directly from biosynthetic processes, or through catalytic dehydration processes of at least one diol.

When said alkenol is a butenol, said diol is preferably a butanediol, more preferably 1,3-butanediol, even more preferably bio-1,3-butanediol, i.e. 1,3-butanediol deriving from biosynthetic processes.

When said diol is 1,3-butanediol, or bio-1,3-butanediol, the diene obtained with the process according to the present disclosure is, respectively, 1,3-butadiene, or bio-1,3-butadiene.

1,3-butadiene is a fundamental product for the petrochemical industry: it represents a substrate for the preparation, among other products, of chloroprene, adiponitrile and hexamethylenediaamine.

In fact, 1,3-butadiene can be used in many industrial sectors including the plastic, synthetic rubber, resin, latex, terpolymer (e.g. ABS), paint and synthetic fibre sectors. The copolymerization product of 1,3-butadiene with styrene is included in the composition of blends for producing tyres.

### BACKGROUND

Currently, more than 95% of the 1,3-butadiene produced on an annual basis is obtained as a sub-product deriving from “steam-cracking” processes for the production of ethylene and other olefins, from which it is separated through extractive distillation where it is formed in small quantities (<5%). 1,3-butadiene can also be obtained from fossil sources through other processes such as, for example, through the catalytic/oxidative dehydrogenation of butane and/or butenes. All of these processes are particularly energy-consuming and imply the emission of high quantities of carbon dioxide (CO₂).

Because of the need to reduce the environmental impact of chemical production, together with the need to use raw materials deriving from renewable sources (for example, biomasses), different processes have been used for the purpose of obtaining bio-1,3-butadiene. Examples of said processes are: dehydration of diols obtained from the fermentation of biomass; direct fermentation of biomass to bio-1,3-butadiene; transformation of ethanol in one or two stages to bio-1,3-butadiene; dehydration followed by dehydrogenation of bio-butanol obtained by fermentation or gasification of biomass.

For example, the dehydration of diols obtained from the fermentation of biomass can be carried out through two consecutive dehydration stages according to the following scheme:

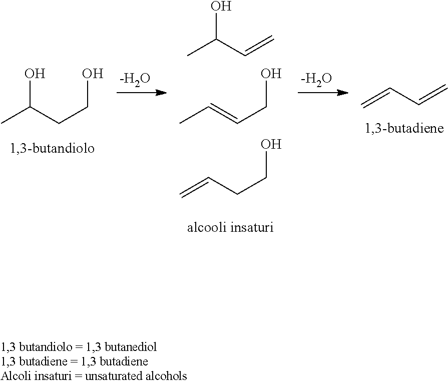

operating as described, for example, by Sato S. et al in the article “Future Prospect of the Production of 1,3-Butadiene from Butanediols”, “Chemistry Letters” (2016), Vol. 45, pp. 1036-1047.

The production of olefins and/or dienes (for example, 1,3-butadiene) by dehydration of unsaturated alcohols, can be carried out in the presence of acid catalysts. The type of acidity, i.e. Bronsted acids or Lewis acids, as well as the force of the acid sites, can be different. The catalytic systems generally used are based on metal oxides such as, for example, silica (Si), aluminium (Al), zirconium (Zr), zinc (Zn), magnesium (Mg), in amorphous form, or based on zeolites. The production of olefins and/or dienes (e.g. 1,3-butadiene) by dehydration of unsaturated alcohols can also be carried out in the presence of cerium-based catalysts, in particular in two-stage processes.

Many efforts have been made in the state of the art in order to identify catalysts suitable for the dehydration of unsaturated alcohols for providing olefins and/or dienes.

For example, English patent GB 1,275,171 describes a process for preparing a lithium phosphate-based catalyst to be used in the dehydration of an epoxide or of a diol to provide a diene. The use of said catalyst in the dehydration of epoxides or of diols to provide dienes is said to enable sub-products to be obtained, mainly carbonyl products that can be reconverted into olefins. Furthermore, said catalyst is said to have the advantage of being able to be calcined at 600° C. without losing its activity so as to be able to be regenerated after use.

American patent U.S. Pat. No. 2,420,477 describes a process for preparing butadiene comprising reacting vinyl ethyl ether with ethylene at a temperature comprised between 125° C. and 250° C., in the presence of a catalyst comprising a “core” of a metal selected from the group consisting of beryllium, magnesium, zinc, cadmium, aluminium, or alloys thereof, said “core” being coated with an oxide of a metal such as, for example, vanadium, niobium, tantalum, chromium, molybdenum, tungsten and uranium. The aforesaid process is said to be carried out at temperatures lower than those normally used for preparing butadiene and substantially to be able to prevent the production of sub-products.

International patent application WO 2013/017496 describes the use of a catalyst comprising a zeolite modified with phosphorus for the dehydration of alcohols to produce olefins with low molecular weight, said catalyst being obtained through a specific process which is said to be easily reproducible to provide a catalyst with good performance levels.

American patent U.S. Pat. No. 4,260,845 describes the dehydration of a saturated alcohol to olefin in the presence of a zinc aluminate-based dehydration catalyst having a ZnO/Al₂O₃ molar ratio of about 1, said catalyst having been heated in air for a sufficient time and at a sufficient temperature to activate it. The aforesaid catalyst is said to have good performance levels in terms of selectivity.

Despite the efforts made in the state of the art, the identification of dehydration catalysts having good performance levels and higher durability, and/or processes that can increase the durability of said catalysts, and/or reduce the formation of sub-products which, as mentioned above, can cause poisoning of the catalyst, is still an objective and therefore of great interest.

It is known that in dehydration reactions of unsaturated alcohols in the presence of acid catalysts, as well as the main product obtained, i.e. the olefin and/or dienes, secondary reactions also take place which lead to the formation of carbonyl compounds such as, for example, aldehydes, ketones, carboxylic acids. Said carbonyl compounds can be formed thanks to the dehydrogenating component present in some dehydration catalysts such as, for example, alumina and silica-aluminas, wherein said component may be more or less marked.

Other secondary reactions that can take place are the oligomerization of olefins and “cracking” phenomena. Said secondary reactions provide compounds that act as precursors of coke and that therefore lead to a deactivation of the dehydration catalyst due to the formation of coke and/or tar that relatively quickly cover the active surface of the catalysts used for the dehydration making it completely inactive.

Deactivation mechanisms of the catalysts are widely known in literature. Detailed descriptions of said mechanisms can be found, for example, in: Petersen Z. and Bell A. T., “Catalyst Deactivation” (1987), Marcel Dekker, INC, New York; Forzatti P. et al, “Catalyst deactivation”, “Catalysis Today” (1999), Vol. 52, pp. 165-181; Bartholomew C. H., “Mechanisms of catalyst deactivation”, “Applied Catalysis A: General” (2001), Vol. 212, pp. 17-60.

Makshina E. V. et al, in the review “Review of old chemistry and new catalytic advances in the on-purpose synthesis of butadiene”, “Chemical Society Reviews” (2014), Vol. 43, pp. 7917-7953, cover the state of the art in relation to the production of 1,3-butadiene from renewable sources and the catalytic systems used for that purpose. The bibliography to which they refer is very extensive (in fact, 246 articles are mentioned) but among the catalysts used, catalysts comprising coke in which said coke is active in catalysis are never mentioned.

There is also vast patent literature related to the production of 1,3-butadiene through the dehydration of unsaturated alcohols, and/or the production of unsaturated alcohols that can be used in the production of 1,3-butadiene and/or the catalytic systems used but, also in this case, catalysts comprising coke in which said coke is active in catalysis are never mentioned. By way of example the following American patents U.S. Pat. Nos. 2,310,809, 2,426,678, 4,400,562, 5,406,007, 6,278,031, 9,434,659; as well as European patent applications EP 3,262,023, EP 3,230,236, EP 3,142,785, and the International patent application WO 2018/073282, all in the name of one of the Applicants, can be mentioned.

A coke active in the catalysis of the conversion reaction of methanol and dimethyl ether (DME) to olefin (MTO E DTO) is described in the article by Chen D. et al, “The Role of Coke Deposition in the Conversion of Methanol to Olefins over SAPO-34”, “Catalyst Deactivation” (1997), Bartholomew C. H. and G. A. Fuentes Eds., Elsevier Science B.V., pp. 159-166. However, in said article, no suggestion is given with respect to the possibility that said coke can be active as a catalyst also in the dehydration reaction of alkenols to produce 1,3-butadiene.

### SUMMARY

The Applicants therefore set out to solve the problem of finding a catalyst comprising coke that can be advantageously used in a process for producing dienes, in particular conjugated dienes, more in particular 1,3-butadiene and even more in particular bio-1,3-butadiene, through the catalytic dehydration of at least one alkenol having a number of carbon atoms greater than or equal to 4, in particular of at least one alkenol deriving from biosynthetic processes, conventionally known as bio-alkenol.

The Applicants have now found a catalyst comprising coke wherein said coke, upon analysis by infrared spectroscopy in diffuse reflection (“Diffuse Reflectance Infrared Fourier Transform Spectroscopy”—DRIFTS), has at least two peaks in a specific wavelength range, which can be advantageously used in a process for the production of dienes. In particular, said catalyst comprising coke, can be advantageously used in a process for producing dienes, in particular conjugated dienes, more in particular 1,3-butadiene and even more in particular bio-1,3-butadiene, through the catalytic dehydration of at least one alkenol having a number of carbon atoms greater than or equal to 4, in particular of at least one alkenol deriving from biosynthetic processes, conventionally known as bio-alkenol. Furthermore, said catalyst comprising coke is able to provide 1,3-butadiene with a high yield and selectivity.

Therefore, the present disclosure provides a catalyst comprising coke characterised in that said coke, upon analysis by infrared spectroscopy in diffuse reflection (“Diffuse Reflectance Infrared Fourier Transform Spectroscopy”—DRIFTS), has at least two peaks at a wavelength comprised between 1450 cm⁻¹ and 1700 cm⁻¹.

The analysis by infrared spectroscopy in diffuse reflection (“Diffuse Reflectance Infrared Fourier Transform Spectroscopy”—DRIFTS) was carried out as described in the following examples.

For the purpose of the present description and the following claims, the definitions of the numerical intervals always comprise the extreme values unless otherwise specified.

For the purpose of the present description and the following claims, the term “comprising” also includes the terms “which essentially consists of” or “which consists of”.

### SUMMARY

In accordance with a preferred embodiment of the present disclosure, said catalyst comprising coke can comprise at least one compound selected, for example, from: aluminium oxide (γ-Al₂O₃), aluminium silicate, silicas-aluminas (SiO₂—Al₂O₃), aluminas, zeolites, metal oxides (for example, lanthanum oxide, zirconium oxide, tungsten oxide, thallium oxide, magnesium oxide, zinc oxide, silver oxide); preferably selected from silicas-aluminas (SiO₂—Al₂O₃).

In accordance with a further preferred embodiment of the present disclosure, said catalyst comprising coke can comprise:
(a) from 2% by weight to 30% by weight, preferably from 6% by weight to 20% by weight, with respect to the total weight of said catalyst, of coke;
(b) from 70% by weight to 98% by weight, preferably from 80% by weight to 94% by weight, with respect to the total weight of said catalyst, of at least one compound selected, for example, from: aluminium oxide (γ-Al₂O₃), aluminium silicate, silicas-aluminas (SiO₂—Al₂O₃), aluminas, zeolites, metal oxides (for example, lanthanum oxide, zirconium oxide, tungsten oxide, thallium oxide, magnesium oxide, zinc oxide, silver oxide); preferably selected from silicas-aluminas (SiO₂—Al₂O₃);
the sum of (a)+(b) being equal to 100.

For the purpose of the present disclosure, said catalyst comprising coke can contain binders such as, for example, alumina, silica, and/or optionally be supported on inert carriers such as, for example, pumice, graphite, silica.

For the purpose of the present description and following claims, the term “zeolites” is to be considered in its widest meaning, i.e. also comprising commonly known materials such as, for example, “zeolite-like”; “zeotype”; zeolites modified with phosphorus, or with a metals such as, for example, sodium, potassium, boron, or with a metal of the lanthanide series; and the like.

The catalyst comprising coke according to the present disclosure can be obtained from the catalytic dehydration reaction of at least one alkenol, preferably 3-buten-2-ol (3-Bu-2-OH) and 2-buten-1-ol (2-Bu-1-OH), providing dienes, preferably providing 1,3-butadiene. In fact, the coke contained in said catalyst is a sub-product that is formed during said dehydration reaction.

Therefore, the present disclosure provides a process for the production of a diene, preferably a conjugated diene, more preferably 1,3-butadiene, comprising the dehydration of at least one alkenol having a number of carbon atoms greater than or equal to 4, in the presence of at least one catalyst comprising coke, characterised in that said coke, upon analysis by infrared spectroscopy in diffuse reflection (“Diffuse Reflectance Infrared Fourier Transform Spectroscopy”—DRIFT), has at least two peaks at a wavelength comprised between 1450 cm⁻¹ and 1700 cm⁻¹.

In accordance with a preferred embodiment of the present disclosure, said alkenol, linear or branched, has general formula CₙH₂ₙO, n being an integer greater than or equal to 4 and less than or equal to 8, preferably greater than or equal to 4 and less than or equal to 6, even more preferably equal to 4.

Specific examples of alkenols that are particularly useful for the purpose of the present disclosure are: 2-buten-1-ol, 3-buten-1-ol, 3-buten-2-ol, 2-methyl-3-buten-2-ol, 4-penten-1-ol, 4-penten-2-ol, 4-penten-3-ol, 3-penten-1-ol, 3-penten-2-ol, 2-penten-1-ol, 5-hexen-1-ol, 5-hexen-2-ol, 5-hexen-3-ol, 5-hexen-4-ol, 4-hexen-1-ol, 4-hexen-2-ol, 4-hexen-3-ol, 3-hexen-1-ol, 3-hexen-2-ol, 2-hexen-1-ol, 2-methyl-3-penten-2-ol, 2-methyl-4-penten-2-ol, 3-methyl-4-penten-2-ol, 6-hepten-1-ol, 6-hepten-2-ol, 6-hepten-3-ol, 6-hepten-4-ol, 6-hepten-5-ol, 5-hepten-1-ol, 5-hepten-2-ol, 5-hepten-3-ol, 5-hepten-4-ol, 4-hepten-1-ol, 4-hepten-2-ol, 4-hepten-3-ol, 3-hepten-1-ol, 3-hepten-2-ol, 2-hepten-1-ol, 7-octen-1-ol, 7-octen-2-ol, 7-octen-3-ol, 7-octen-4-ol, 7-octen-5-ol, 7-octen-6-ol, 6-octen-1-ol, 6-octen-2-ol, 6-octen-3-ol, 6-octen-4-ol, 6-octen-5-ol, 5-octen-1-ol, 5-octen-2-ol, 5-octen-3-ol, 5-octen-4-ol, 4-octen-1-ol, 4-octen-2-ol, 4-octen-3-ol, 3-octen-1-ol, 3-octen-2-ol, 2-octen-1-ol.

In accordance with a further preferred embodiment of the present disclosure, said alkenol has a number of carbon atoms equal to 4 and is therefore a butenol.

In accordance with a further preferred embodiment of the present disclosure, said butenol can be selected from 2-buten-1-ol (crotyl alcohol) (2-Bu-1-OH), 3-buten-2-ol (methyl-vinyl-carbinol) (3-Bu-2-OH), 3-buten-1-ol (allyl carbinol) (3-Bu-1-OH), or mixtures thereof, and even more preferably, from 2-buten-1-ol (2-Bu-1-OH), 3-buten-2-ol (3-Bu-2-OH), or mixtures thereof.

It is to be noted that when the alkenol can exist in different enantiomeric or stereoisomeric forms, it is implied that the process according to the present disclosure can be carried out with either of these types, both in purified form and in mixture.

For example, in the aforesaid process, the E isomer (trans) of 2-buten-1-ol (2-Bu-1-OH), the Z isomer (cis) of 2-buten-1-ol (2-Bu-1-OH), or a mixture of said two isomers, can be used indifferently. Likewise, in the aforesaid process, the enantiomer (R) of 3-buten-2-ol (3-Bu-2-OH), the enantiomer (S) of 3-buten-2-ol (3-Bu-2-OH), or a racemic mixture of said two enantiomers, can be used.

In accordance with a preferred embodiment of the present disclosure, said alkenol having a number of carbon atoms greater than or equal to 4, can be obtained directly from biosynthetic processes, or through catalytic dehydration processes of at least one diol.

In accordance with a preferred embodiment of the present disclosure, when said alkenol has a number of carbon atoms equal to 4, and is therefore a butenol, said butenol can be obtained by means of the catalytic dehydration of a butanediol, preferably 1,3-butanediol, in the presence of a cerium oxide-based catalyst, wherein said cerium oxide-based catalyst is obtained through precipitation, in the presence of at least one base, of at least one compound containing cerium. Further details related to said process can be found, for example, in International patent application WO 2015/173780 in the name of one of the Applicants and incorporated herein for reference purposes.

In accordance with a preferred embodiment of the present disclosure, said diol, preferably a butanediol, more preferably 1,3-butanediol, can derive from the fermentation of sugars, preferably from the fermentation of sugars deriving from biomass.

For the purposes of the present description and the following claims, the term “biomass” indicates any organic material of plant origin that comprises: products deriving from agriculture such as, for example, plants and parts of plants of guayule, thistle, corn, soy, cotton, flax, rape, sugar cane, palm, including scraps, residues and wastes deriving from said products or from their processing; products deriving from crops of plant species expressly cultivated for energy use such as, for example, miscanthus, panicum, common cane, including scraps, residues and wastes deriving from said products or from their processing; products deriving from forestation or from forestry, including scraps, residues and wastes deriving from said products or from their processing; scraps from agri-food products intended for human food or for livestock; residues from the paper industry; wastes from the separate collection of solid urban waste such as, for example, scraps of fruit and vegetables, paper.

In accordance with a particularly preferred embodiment of the present disclosure, said diol, preferably a butanediol, more preferably 1,3-butanediol, derives from the fermentation of sugars deriving from biomass of guayule and/or thistle, including scraps, residues and wastes deriving from guayule and/or thistle, or from their processing. Even more preferably, said diol, preferably a butanediol, more preferably 1,3-butanediol, derives from the fermentation of sugars deriving from guayule biomass, including scraps, residues and wastes deriving from guayule, or from its processing.

For the purpose of producing the aforesaid sugars, said biomass can be subjected to physical treatments (for example, extrusion, “steam explosion”, and the like) and/or to chemical hydrolysis and/or to enzymatic hydrolysis, obtaining mixtures of carbohydrates, of aromatic compounds and of other products that derive from the cellulose, hemicellulose and lignin present in the biomass. In particular, the carbohydrates obtained are mixtures of glucides with 5 and 6 carbon atoms that comprise, for example, sucrose, glucose, xylose, arabinose, galactose, mannose, fructose, which will be used in fermentation. Processes related to the production of sugars from biomass, in particular sugars from lignocellulose biomass, are described in the state of the art such as, for example, in International patent application WO 2015/087254, in the name of one of the Applicants and incorporated herein for reference purposes.

Further biotechnological processes for obtaining bio-1,3-butanediol, starting from renewable sources, are described, for example, in American patent U.S. Pat. No. 9,017,983, and in American patent applications US 2012/0329113 and US 2013/0109064.

When the diol, preferably a butanediol, more preferably 1,3-butanediol, derives from biosynthetic processes, for example from the fermentation of sugars as described above, said diol is generally obtained in the form of an aqueous mixture. Before proceeding with the catalytic dehydration of the diol which leads to obtaining the corresponding alkenol, it is possible to subject the aforesaid aqueous mixture, comprising said diol obtained from biosynthetic processes, to common separation processes such as, for example, total or partial distillation of water and of the diol contained in said mixture. In fact, said aqueous mixture, after filtration and deionization, can be advantageously used as such in the catalytic dehydration process which leads to the corresponding alkenol being obtained, without the need to subject it to expensive water removal processes or however limiting such removal.

In turn, the alkenol can be obtained in the form of an aqueous mixture.

Said aqueous mixture can be subjected to distillation, to recover the alkenol, pure or in the form of an azeotrope with water, or used as such.

For example, the catalytic dehydration of 1,3-butanediol leads to a mixture of butenols [2-buten-1-ol (2-Bu-1-OH), 3-buten-2-ol (3-Bu-2-OH), 3-buten-1-ol (3-Bu-1-OH)] being obtained, which can be separated through distillation in the form of minimum azeotropes with water. The azeotropic mixtures of butenols can be used as such, mixed together, or mixed together with butenols added, in mixture or individually, or with water added, in order to be used in the catalytic dehydration process of the present disclosure for producing 1,3-butadiene.

In accordance with an embodiment of the present disclosure, in the aforesaid process for the production of a diene, said alkenol having a number of carbon atoms greater than or equal to 4, can be mixed with a diluent which can be selected, for example, from an inert gas such as, for example, nitrogen (N₂), argon (Ar), preferably N₂; or it can be mixed with a compound having a boiling temperature comprised between 25° C. and 150° C. under normal conditions and preferably, a boiling temperature comprised between 50° C. and 125° C. under normal conditions, and a melting temperature less than or equal to 20° C. under normal conditions, which can be selected, for example, from water, tetrahydrofuran, cyclohexane, benzene, or mixtures thereof. Nitrogen (N₂) and water are preferred, and water is particularly preferred.

It is important to note that said water may be residual water deriving from the biosynthetic process used for producing said at least one alkenol.

In accordance with a preferred embodiment of the present disclosure, said process for the production of a diene can be carried out, in the case in which the diluent is selected from inert gases, with a molar ratio between diluent and alkenol (or alkenols) greater than 0.3, preferably comprised between 0.5 and 2.

In accordance with a preferred embodiment of the present disclosure, said process for the production of a diene can be carried out, in the event in which the diluent is selected from compounds having a boiling temperature comprised between 25° C. and 150° C. under normal conditions, preferably comprised between 50° C. and 125° C. under normal conditions and a melting temperature less than or equal to 20° C. under normal conditions, with a molar ratio between diluent and alkenol (or alkenols) comprised between 0.01 and 100, preferably comprised between 0.1 and 50, more preferably comprised between 1 and 10.

In accordance with a preferred embodiment of the present disclosure, said process for the production of a diene can be carried out at a temperature comprised between 250° C. and 500° C., preferably comprised between 280° C. and 450° C.

In accordance with a preferred embodiment of the present disclosure, said process for the production of a diene can be carried out at a pressure comprised between 5 kPa and 5000 kPa, preferably comprised between 30 kPa and 350 kPa, more preferably comprised between 80 kPa and 250 kPa.

The process for the production of a diene according to the present disclosure can be carried out in the gaseous phase or in the mixed liquid/gas phase.

In accordance with an embodiment of the present disclosure, the aforesaid process for the production of a diene is carried out in the gaseous phase.

Said process for the production of a diene can be carried out in any type of reactor, preferably a fixed bed reactor, a mobile bed reactor, or a fluidized bed reactor.

In accordance with an embodiment of the present disclosure, said process for the production of a diene can be carried out in a fixed bed reactor.

In the case in which a fixed bed reactor is used, the catalyst comprising coke can be split across various beds.

The reactor set-up can comprise the recycling of part of the reaction effluents or of the catalytic material, in a “recirculated” reactor configuration.

In accordance with an alternative embodiment of the present disclosure, when the process for the production of a diene is carried out in a mixed liquid/gas phase, a Continuous flow Stirred Tank Reactor (CSTR) can be used, containing the catalyst comprising coke in dispersion.

It is important to note that when in the process for the production of a diene object of the present disclosure at least one alkenol is used which derives from the catalytic dehydration of at least one diol, however the latter is obtained, the dehydration of said at least one diol to provide the at least one alkenol and the subsequent dehydration of said at least one alkenol to provide the diene can be carried out:
in the same reactor or in different reactors, and preferably in different reactors;
continuously or in batches, and preferably continuously.

The process for the production of a diene according to the present disclosure can be carried out continuously also in a reactor configuration which envisages at least two reactors in parallel, preferably two fixed-bed reactors in parallel wherein, when a reactor is operating, in the other reactor the catalyst comprising coke can be regenerated.

When the process for the production of a diene is carried out continuously, the space velocity WHSV (“Weight Hourly Space Velocity”), i.e. the ratio between the quantity by weight of reactant fed to the reactor and the quantity by weight of the catalyst comprising coke in the reactor itself, can be comprised between 0.5 h⁻¹ and 10 h⁻¹, preferably comprised between 1 h⁻¹ and 5 h⁻¹.

The contact time (τ), calculated as the ratio between the volume of catalyst comprising coke loaded into the dehydration reactor and the volumetric supply flow rate in the reaction conditions, is preferably comprised between 0.01 seconds and 10 seconds, more preferably comprised between 0.05 seconds and 8 seconds, even more preferably comprised between 0.1 seconds and 4 seconds.

As mentioned above, the catalyst comprising coke object of the present disclosure, may be obtained from the catalytic dehydration reaction of at least one alkenol, preferably 3-buten-2-ol (3-Bu-2-OH) and 2-buten-1-ol (2-Bu-1-OH).

Therefore, the present disclosure further provides a process for the production of a catalyst comprising coke, characterised in that said coke, upon analysis by infrared spectroscopy in diffuse reflection (“Diffuse Reflectance Infrared Fourier Transform Spectroscopy”—DRIFTS) has at least two peaks at a wavelength comprised between 1450 cm⁻¹ and 1700 cm⁻¹, said process comprising the dehydration of a mixture of alkenols comprising 3-buten-2-ol (3-Bu-2-OH) and 2-buten-1-ol (2-Bu-1-OH), in the presence of at least one compound selected from: aluminium oxide (—Al₂O₃), aluminum silicate, silicas-aluminas (SiO₂—Al₂O₃), aluminas, zeolites, metal oxides (for example, lanthanum oxide, zirconium oxide, tungsten oxide, thallium oxide, magnesium oxide, zinc oxide, silver oxide), preferably selected from silicas-aluminas (SiO₂—Al₂O₃), said process being carried out at a temperature comprised between 250° C. and 500° C., preferably comprised between 280° C. and 450° C., for a time comprised between 2 hours and 10 hours, preferably comprised between 2.5 hours and 9 hours.

It is to be noted that the operating conditions of the aforesaid process for the production of a catalyst comprising coke, not specifically indicated, for example, the space velocity WHSV (“Weight Hourly Space Velocity”), the contact time (i), the types of reactors, etc., are the same as those described above in the case of the process for the production of a diene, a further object of the present disclosure.

It is to be noted that, for the purpose of demonstrating the presence of the aforesaid peaks, in the following example, three catalysts comprising coke, obtained through the dehydration of the individual butenols, i.e. 2-buten-1-ol (2-Bu-1-OH), 3-buten-2-ol (3-Bu-2-OH), 3-buten-1-ol (3-Bu-1-OH), were subjected to analysis by infrared spectroscopy in diffuse reflection (“Diffuse Reflectance Infrared Fourier Transform Spectroscopy”-DRIFTS).

For the purpose of putting the present disclosure into practice and illustrating it more clearly, below are some non-limiting examples.

### EXAMPLE 1

### (i) Preparation of a Mixture of Raw Butenols

For the purpose, a mixture of 1,3-butanediol (1,3-BDO) was carried out, having a concentration by weight equal to 83% of 1,3-butanediol (1,3-BDO), 17% of water (mixture 1), respectively, which was then used for the dehydration reaction.

The reactor in which said dehydration reaction was carried out was comprised of an AISI 304 stainless steel tubular element of height (h) equal to 260 mm and internal diameter (Φ) equal to 10 mm, preceded and connected to an evaporator, both provided with electric heating. The outlet of the reactor was instead connected to a first condenser connected to a receiving flask, and operating at 15° C., for the purpose of enabling the recovery of the products obtained from the first dehydration reaction in the form of liquid at room temperature (25° C.) in said receiving flask. Said receiving flask was in turn connected to a sampling system comprising a steel cylinder of volume (V) equal to 300 ml and provided at both ends with interception valves. The vapours/gases deriving from the first dehydration reaction and optionally not condensed in the system previously described could further flow through the aforesaid steel cylinder connected in turn to a volumetric meter that therefore measured the quantity thereof.

The products obtained, both in the form of liquid, and in the form of vapour/gas, were characterized by gas chromatography, using:
for the products in liquid form, a Thermo Trace gas chromatograph, equipped with an FID detector and AQUAWAX column (Grace 30 m length×0.53 mm internal diameter×1.0 μm film thickness);
for the products in gas form, a 490 micro GC Varian/Agilent gas chromatograph with four channels equipped with the following columns: Pora Plot Q length 10 m, MolSieve 5 Å length 4 m, Al₂O₃ length 10 m with “backflush” functionality, CPSil-19 CB length 7.5 m.

The catalyst used in said dehydration reaction was a Cerium oxide (CeO₂) based material in granules of a size comprised between 0.5 mm and 1 mm and it was loaded into the aforesaid reactor in a quantity equal to 10 g (3.5 ml). Said catalyst was prepared according to the laboratory procedure described below.

For that purpose, 500 g of a commercial aqueous solution of about 30% ammonium hydroxide (NH₄OH), (28%-30% NH₃ Basis ACS reagent Aldrich) were added to a first 3 litre beaker provided with a half-moon stirrer blade made of teflon, with 500 g of water, and an electrode was introduced for measuring the pH [Metrohm glass electrode for measuring the pH (6.0248.030), connected to the Metrohm 780 pH meter]. In a second 2 litre beaker provided with a magnetic anchor stirrer, a 100 g cerium nitrate hexahydrate solution (99% Aldrich) was prepared in 1000 g of water: the cerium nitrate hexahydrate was then dissolved through vigorous stirring at room temperature (25° C.). The solution obtained was inserted into a dropping funnel and fed drop by drop, in 2 hours, to the ammonium hydroxide solution described above, contained in the 3 litre beaker, with constant vigorous stirring. The pH of the suspension obtained was equal to 10.2. The solid in suspension was filtered, washed with 2 litre of water, and then dried in a stove, at 120° C., for 2 hours. The synthesis was repeated until 2000 g of solid were obtained.

1270 g of the solid thus obtained, subject to screening at 0.125 mm, were inserted into an extruder to which 175.9 g of 25% ammonium hydroxide (NH₄OH) solution were also added (obtained by diluting the solution at 28%-30% NH₃ Basis ACS reagent Aldrich) using a Watson Marlow peristaltic pump set to 5 rpm. After said addition, 158 g of demineralized water were also added, thus providing the right consistency for extrusion. The “pellets” obtained at the outlet of the extruder were dried in air and, subsequently, a 100 g portion was calcined at 800° C. with a 1° C./minute ramp to 800° C., followed by isotherm in temperature for 6 hours. The calcined solid was granulated and screened and the fraction of granules of size comprised between 0.5 mm and 1 mm was used as a catalyst.

Said dehydration reaction was then carried out by supplying the mixture 1, first to the aforesaid evaporator previously heated to a temperature of 250° C., and from this to the aforesaid tubular reactor previously heated so as to have an internal temperature during the dehydration reaction equal to 400° C. Both the evaporator and the reactor were kept at atmospheric pressure (1 bar).

The flow rate of the mixture 1 supplied to the evaporator was equal to 100 g/h, whereas the flow rate to the reactor, expressed as WHSV, was equal to 10 h⁻¹.

The test was carried out for a sufficient time for collecting a suitable quantity of raw product.

### (ii) Purification of Raw Butenols

The mixture of raw butenols obtained as described above was subjected to a first purification, through distillation, for the purpose of removing the unreacted 1,3-butanediol (1,3-BDO). It is to be noted that the butenols present in said mixture form, with water, azeotropic mixtures for which it is not possible through simple distillation to separate them from the water in order to obtain them pure.

The distillation was carried out at atmospheric pressure, adding to said mixture contained in the boiler, 3,5-di-tert-4-butylhydroxytoluene (BHT) so as to have a concentration thereof in said mixture equal to about 200 ppm. Said distillation was carried out using a 40-plate Oldershaw column (2 sections with 20 plates), loading said mixture into the boiler in a single batch and taking various head samples on the basis of the temperatures recorded, gradually concentrating the boiler of the heavier components. The distillation conditions (reflux ratio, boiler heating power, quantity of distillate taken) were varied as a function of the boiling temperatures of the species to be separated and the head temperatures recorded.

The distillation conditions are shown in Table 1.

TABLE 1
Distillation conditions of alkenols at atmospheric pressure

| | ΔT boiler | ΔT head | |
|---|---|---|---|
| | (° C.) | (° C.) | RR⁽¹⁾ |
| Load | — | — | — |
| Fraction 1 | 101.3-103.0 | 56.0-84.5 | 100 |
| Fraction 2 | 103.1-104.3 | 85.0-86.8 | 100-30 |
| Fraction 3 | 104.9-133.6 | 86.4-87.1 | 30 |
| Fraction 4 | 114.6-152.3 | 87.1-94.4 | 30-40 |
| Fraction 5 | 154.3-167.9 | 93.6-119.4 | 40-60 |
| Fraction 6 | 169.2-208.6 | 120.1-121.2 | 60-70 |
| Fraction 7 | 208.6-210.8 | 121.2-130.1 | 70 |
| Boiler | — | — | — |

⁽¹⁾reflux ratio.

In particular:
Fraction 1 (up to about 84° C.) corresponds to the lightest part to be removed;
Fraction 2 and Fraction 3 correspond to an azeotrope at T=86.5° C.-87° C. between the lowest boiling point alkenol, i.e. 3-buten-2-ol (methylvinylcarbinol) (3-Bu-2-OH) and water (said azeotrope having composition: 73% by weight of 3-buten-2-ol (3-Bu-2-OH) and 27% by weight of water);
in Fraction 4, 2-buten-1-ol (crotyl alcohol in the cis and trans forms) (2-Bu-1-OH) and the small portion of 3-buten-1-ol (allyl carbinol) (3-Bu-1-OH) together with 35% by weight of water, also start to distillate;
in Fraction 5 the water runs out and therefore the temperature rises to about 120° C.;
Fraction 6 and Fraction 7 correspond to 2-buten-1-ol (crotyl alcohol) (2-Bu-1-OH) at 95%-97%.

The fractions containing butenols were joined in a single fraction that represents the mixture used for producing the catalyst comprising coke according to the present disclosure. The composition is described in Table 2.

TABLE 2

| | |
|---|---|
| Water | 37.0% |
| Light⁽¹⁾ | 0.1% |
| 2-Bu-1-OH + 3-Bu-2-OH | 60.3% |
| 3-Bu-1-OH | 0.2% |
| Medium boiling point⁽²⁾ | 2.2% |
| Heavy⁽³⁾ | 0.2% |

⁽¹⁾lightest compounds of low boiling point butenol, i.e. 3-buten-2-ol (3-Bu-2-OH) (T_{BOILING} = 97° C.);
⁽²⁾lightest compounds of high boiling point butenol, i.e. 2-buten-1-ol (2-Bu-1-OH) (T_{BOILING} = 121.5° C.); heavier than low boiling point alkenol, i.e. 3-buten-2-ol (3-Bu-2-OH) (T_{BOILING} = 97° C.), excluding 1,3-butadiene (1,3-BDE) [includes the medium boiling point 3-buten-1-ol (3-Bu-1-OH) (T_{BOILING} = 113.5)];
⁽³⁾heavier compounds of the high boiling point butenol, i.e. 2-buten-1-ol (2-Bu-1-OH) (T_{BOILING} = 121.5° C.).

### EXAMPLE 2

### Formation of the Catalyst Comprising Coke and Production of 1,3-butadiene (1,3-BDE)

A first test was carried out by supplying the mixture of butenols obtained as described in Example 1 and reported in Table 2, in a fixed-bed tubular reactor—PFR (“Plug Flow Reactor”) made of AISI 316L stainless steel, with a length equal to 400 mm and diameter equal to 9.65 mm, loaded with 0.6 g of silica-alumina, containing 3.8% of Al and obtained as described below.

7.55 g of aluminium tri-sec-butoxide (Aldrich) as an alumina precursor (Al₂O₃) were inserted into a first 500 ml round-bottom flask, and 50.02 g of orthosilicic acid (Aldrich, <20 mesh), as a silica precursor (SiO₂), were inserted into a second 500 ml round-bottom flask, with 250.02 g of demineralized water. The suspension of orthosilic acid obtained was added slowly (10 min) to said first round-bottom flask containing aluminium tri-sec-butoxide, and the mixture obtained was kept at 90° C., for about 1 hour, with vigorous stirring (500 rpm). After cooling to room temperature (25° C.), the suspension obtained was filtered and the solid obtained was washed with 5 litres of demineralized water, dried at 120° C. for one night and subsequently calcined at 500° C. for 5 hours, obtaining a colourless powder (47.95 g) (defined as the “active phase”).

Part of the aforesaid “active phase, 40.42 g, was mixed with 24.43 g of pseudoboehmite Versal™ V-250 (UOP), as an alumina precursor (Al₂O₃) of the binder, and 302 ml of a 4% acetic acid solution, in a 800 ml beaker. The mixture obtained was kept, under agitation, at 60° C., for 2 hours. Then, the beaker was transferred to a heating plate and, with vigorous stirring, the mixture was heated for a night to 150° C. until dry. The solid obtained was then calcined at 550° C. for 5 hours, obtaining 60.45 g of a colourless product which was mechanically granulated and the fraction of granules of dimensions comprised between 0.1 mm and 1.0 mm was used as a dehydration catalyst in said tubular reactor.

The catalyst prepared as described above (0.6 g) was inserted into the reactor using quartz wool as a support for the granules so that they were arranged in the isotherm area of the reactor. The reactor was set with a down flow arrangement.

The catalyst was pre-treated in situ at 300° C. under nitrogen (N₂) flow.

The supply of the aforesaid mixture of butenols was carried out from the top of the reactor, at atmospheric pressure (0.1 MPa), through vaporization, so as to allow the reactants to reach the reaction temperature before entering into contact with the catalyst, through the use of an infusion pump and a 5 mL Hamilton pump connected to a heated steel line and subsequently sent to the reactor, under nitrogen (N₂) flow: the total volumetric flow rate was equal to 28 mL/min of which 2% by volume of said mixture of butenols. Said reactor was heated to a temperature of 300° C. using an electric oven and the temperature was controlled by an integrated thermocouple connected to the temperature controller of the electric oven and a second axial thermocouple, placed inside said tubular reactor, which indicates the real temperature of the catalytic bed and was selected as the reference temperature.

The temperature of the tubular reactor was maintained at 300° C. as described above and the contact time (τ) was equal to 0.67 seconds.

Downstream of the reactor, the products obtained were sampled in a gas chromatograph (GC) online for analysis. The online analysis of the gases was carried out through an Agilent HP6890 gas chromatograph (GC) equipped with two capillary columns: an HP-5 column (“crosslinked” 5% phenyl methyl siloxane) 50 m in length, 0.2 mm diameter, 0.33 micron of film, connected to a flame ionization detector (FID) and a HP-Plot-Q column (“bonded” polistyrene divinyl benzene) 30 m in length, 0.32 mm diameter, 20 micron of film, connected to a thermal conductivity detector (TCD). The carrier used in both columns was helium with a flow equal to 0.8 mL/min (HP-5 column) and equal to 3.5 mL/min (HP-Plot-Q column).

### DETAILED DESCRIPTION OF THE DRAWINGS

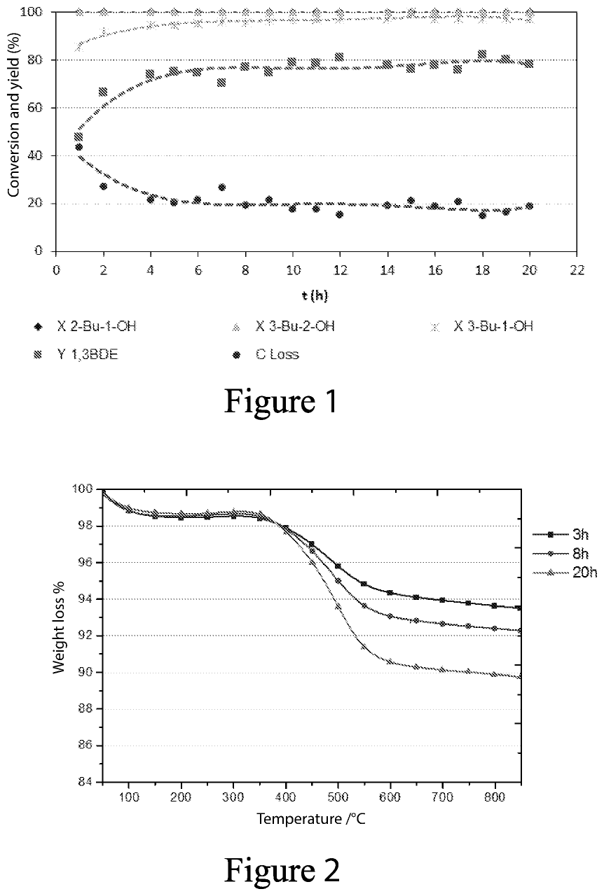

The results are shown in FIG. 1 [in the ordinate the conversion and the yield as a percent (%) were reported; in the abscissa the reaction time in hours (h) was reported].

The catalytic performance levels shown in FIG. 1 are expressed by calculating the conversion of the alkenols (C_{ALCH.}), the selectivity to 1,3-butadiene (S_{1,3-BDE}) and the yield to 1,3-butadiene (R_{1,3-BDE}) according to the formulae reported below:

$${C}_{ALCH} = \frac{{( {moli}_{ALCH} )}_{in} - {( {moli}_{ALCH} )}_{out}}{{( {moli}_{ALCH} )}_{in}} \times 100$$

$${S}_{1 , 3 - BDE} = \frac{{moli}_{1 , 3 - BDE}}{{( {moli}_{ALCH .} )}_{in} - {( {moli}_{ALCH .} )}_{out}} \times 100$$

$${R}_{1 , 3 - BDE} = \frac{{C}_{ALCH .} \times {S}_{1 , 3 - BDE}}{100}$$

wherein:
moli_{ALCH}=moles of alkenols [referring to 3-buten-2-ol (3-Bu-2-OH) and to 2-buten-1-ol (2-Bu-1-OH)];
(moli_{ALCH.})ᵢₙ=moles of alkenols at the inlet;
(moli_{ALCH.})ₒᵤₜ=moles of alkenols at the outlet;
mol_{1,3-BDE}=moles of 1,3-butadiene (1,3-BDE) produced.

From the data reported in FIG. 1, it can be deduced that the yield to 1,3-butadiene (Y 1,3BDE) starts from an initial value of about 47%, settling after 4 hours to about 70%. After 4 hours, the yield to 1,3-butadiene (Y 1,3BDE) continues to increase slowly until stabilizing at an average value of 80% after 10 hours. FIG. 1 also shows the trend of the carbon loss (C loss) that represents the indicator of the coke that gradually forms on the catalyst. In FIG. 1:
X 2-Bu-1-OH refers to the conversion of 2-buten-1-ol (2-Bu-1-OH);
X 3-Bu-2-OH refers to the conversion of 3-buten-2-ol (3-Bu-2-OH);
X 3-Bu-1-OH refers to the conversion of 3-buten-1-ol (3-Bu-1-OH).

For the purpose of better highlighting the role of coke on the increased yield, the aforesaid Example 1 was repeated 3 times, interrupting the reaction after 3 h, 8 h and 20 h. The catalysts unloaded from the reactor at said times, were subjected to Thermal Gravimetric Analysis (TGA) in air to calculate the amount of coke adsorbed. Said analysis was carried out using an SDT Q 600 instrument (TA Instruments), loading 15 mg of catalyst, in air flow (100 mL/min) and with a temperature ramp (10° C./min until 900° C. and subsequent isotherm at 900° C. for 5 minutes) so as to quantify the organic residues present. The results are shown in FIG. 2 [in the ordinate the weight loss as a percentage (%) was reported; in the abscissa the temperature in degrees centigrade (° C.) was reported]. From FIG. 2, considering the weight loss connected with the presence of coke (temperatures greater than 380° C.), it can be deduced that there is rapid formation of coke in the first 3 h (5% weight loss): to this time interval corresponds the fastest increase in yield up to pre-settlement values (as shown in FIG. 1). The other two curves of the TGA show that at values greater than 3 h the coke continues to be formed but more slowly: to this condition corresponds the settlement at maximum yield values (as shown in FIG. 1) which takes place slowly up to 10 hours before stabilizing completely.

### EXAMPLE 3

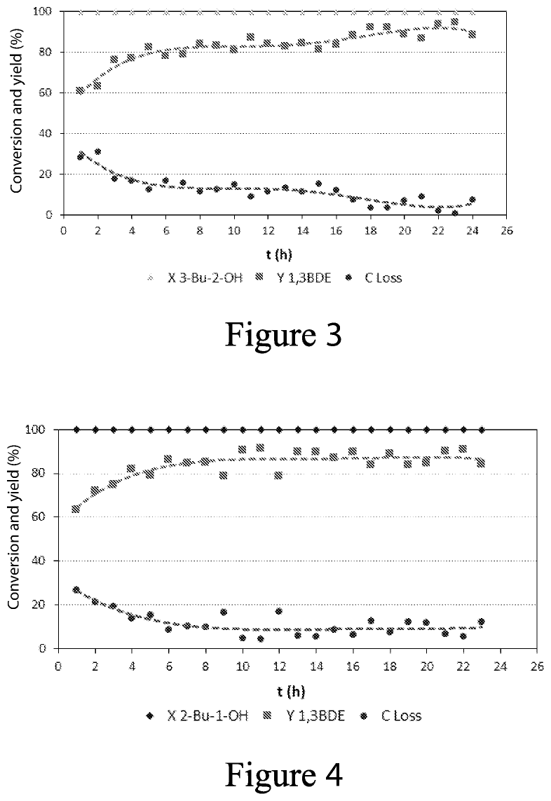

For the purpose of verifying which is the type of catalyst comprising coke active in the catalysis, Example 2 was repeated but instead of using the mixture of butenols it uses the individual isomers supplied separately, i.e. 3-buten-2-ol (3-Bu-2-OH), 2-buten-1-ol (2-Bu-1—OH) and 3-buten-1-ol (3-Bu-1-OH). The conditions used were the same as in Example 2. From these tests it can be deduced that both 3-buten-2-ol (3-Bu-2-OH) [FIG. 3 in which in the ordinate the conversion and yield as a percentage (%) were reported; in the abscissa the time in hours (h) was reported], and 2-butenl-ol (2-Bu-1-OH) [FIG. 4 in which in the ordinate the conversion and yield as a percentage (%) were reported; in the abscissa the time in hours (h) was reported], have an analogous yield profile to that of the mixture of butenols used in Example 2, as shown in FIG. 1 (rapid increase in yield between 0 and 3 hours and then a further increase until settlement at the maximum values after 10 hours).

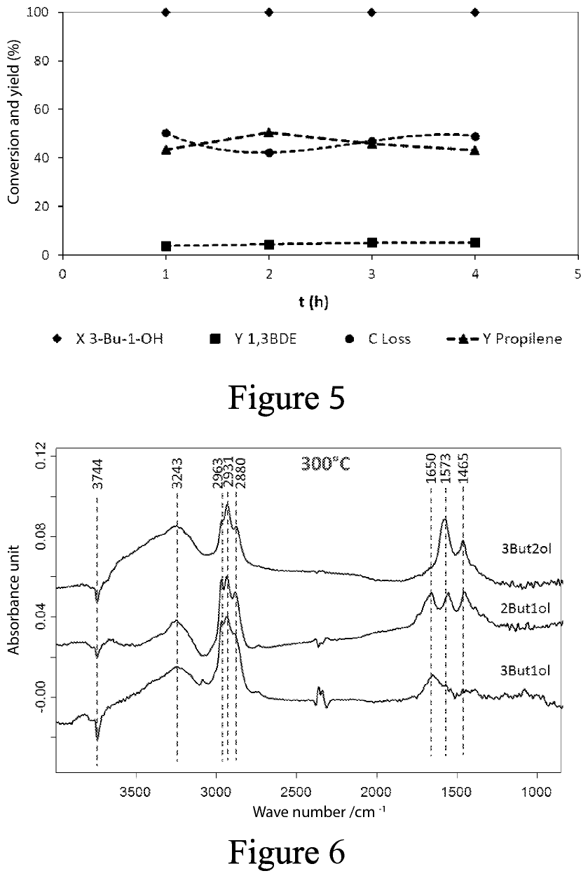

The behaviour of 3-buten-1-ol (3-Bu-1-OH) is completely different [FIG. 5 in which in the ordinate the conversion and yield as a percentage (%) were reported; in the abscissa the time in hours (h) was reported], which mainly produces propylene (40%-50% in yield) and abundant coke (C loss).

### In FIG. 3:

X 3-Bu-2-OH refers to the conversion of 3-buten-2-ol (3-Bu-2-OH);
C loss refers to the trend of the “carbon loss”.

### In FIG. 4:

X 2-Bu-1-OH refers to the conversion of 2-buten-1-ol (2-Bu-2-OH);
C loss refers to the trend of the “carbon loss”.

### In FIG. 5:

X 3-Bu-1-OH refers to the conversion of 3-buten-1-ol (3-Bu-1-OH).

The catalysts comprising coke formed by the three isomers were subjected to analysis by infrared spectroscopy in diffuse reflection (“Diffuse Reflectance Infrared Fourier Transform Spectroscopy”—DRIFTS) operating as follows.

For that purpose, three samples of fresh catalyst were prepared (i.e. 0.6 g of silica-alumina containing 3.8% di Al) operating as described in Example 2. Each sample was diluted in potassium bromide (KBr) in the ratio of 1:10, treated at 450° C. in helium (He) for 20 minutes and then cooled to 50° C.: then a “single pulse” of the reactant was carried out, i.e. of the isomer 3-buten-2-ol (3-Bu-2-OH) (first sample), 2-buten-1-ol (2-Bu-1-OH) (second sample) and 3-buten-1-ol (3-Bu-1-OH) (third sample) on the sample and then, subsequently, the temperature was increased and the spectrum was recorded at 300° C. The aforesaid analysis was carried out using a FT-IR Bruker Vertex 70 spectrophotomer equipped with a Pike DiffusIR cell and with a MCT detector: the spectra were recorded and processed using the processing of data acquired through OPUS software. The analysis parameters were as follows:
scanning interval: 4000 cm⁻¹-450 cm⁻¹;
number of scans per sample: 32;
background: spectrum recorded by positioning the starting catalyst (without coke) in the accessory housing;
spectral resolution: 4 cm⁻¹;
regulation of the mirrors: 3.5 mm (the mirrors of the DRIFTS accessory were aligned so that the instrument detector could detect the maximum absorbance).

The results obtained are shown in FIG. 6 in which in the ordinate the absorption in absorbance units was reported; in the abscissa the wave number in cm⁻¹ was reported. From the spectrum reported in FIG. 6 it can be deduced that effectively in the case of the two isomers that are mainly formed 1,3-butadiene (1,3-BDE), i.e. 3-buten-2-ol (3-Bu-2-OH) (first sample—indicated in FIG. 6 as 3But2ol) and 2-buten-1-ol (2-Bu-1-OH) (second sample—indicated in FIG. 6 as 2ButIol), the coke of the catalyst comprising coke obtained has two characteristic bands at 1465 cm⁻¹ and 1573 cm⁻¹, whereas the isomer 3-buten-1-ol (3-Bu-1-OH) (third sample—indicated in FIG. 6 as 3But1ol) (which leads to the formation of propylene and coke as reported in FIG. 5) has a band at 1650 cm⁻¹ confirming the different nature of the two types of coke. Said bands are attributed to the stretching C═C and C—H of aromatic type (“coke bands”) as reported in literature by Ibarra A. et al, in the article “Dual coke deactivation pathways during the catalytic cracking of raw bio-oil and vacuum gasoil in FCC conditions”, “Applied Catalysis B: Environmental” (2016), Vol. 182, pp. 336-346.

### EXAMPLE 4

For comparative purposes, Example 2 was repeated only supplying 3-buten-1-ol (3-Bu-1-OH), for 4 hours.

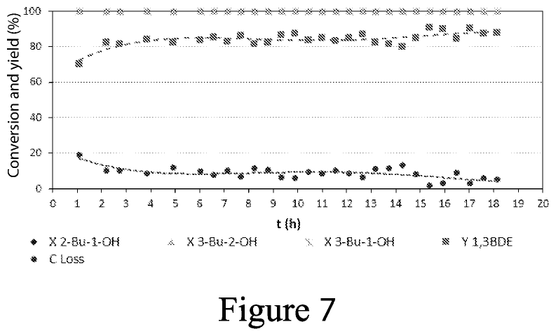

After 4 hours, the 3-buten-1-ol (3-Bu-1-OH), was replaced with the same mixture of butenols used in Example 2 (i.e. the mixture of butenols obtained as described in Example 1 and reported in Table 2). The results are shown in FIG. 7 in which in the ordinate the conversion and the yield as a percent (%) were reported; in the abscissa the time in hours (h) was reported. From FIG. 7 it can be deduced that the coke formed by 3-buten-1-ol (3-Bu-1-OH) has no effect on the promotion of the yield of catalyst: in fact, only after the replacement, in the supply, of 3-buten-1-ol (3-Bu-1-OH), with the same mixture of butenols used in Example 2, can an increase in the yield to 1,3-butadiene (Y 1,3BDE) be observed.

### In FIG. 7:

X 2-Bu-1-OH refers to the conversion of 2-buten-1-ol (2-Bu-1-OH);
X 3-Bu-2-OH refers to the conversion of 3-buten-2-ol (3-Bu-2-OH);
X 3-Bu-1-OH refers to the conversion of 3-buten-1-ol (3-Bu-1-OH);
C loss refers to the trend of the “carbon loss”.

1. A catalyst comprising coke wherein said coke, upon analysis by infrared spectroscopy in diffuse reflection, has at least two peaks at a wavelength comprised between 1450 cm⁻¹ and 1700 cm⁻¹.

2. The catalyst comprising coke according to claim 1, wherein said catalyst comprises at least one compound selected from the group consisting of: aluminum oxide (γ-Al₂O₃), aluminum silicate, silicas-aluminas (SiO₂—Al₂O₃), aluminas, zeolites, and metal oxides (such as lanthanum oxide, zirconium oxide, tungsten oxide, thallium oxide, magnesium oxide, zinc oxide, silver oxide).

3. The catalyst comprising coke according to claim 1, wherein said catalyst comprises:
(a) from 2% by weight to 30% by weight, with respect to the total weight of said catalyst, of coke;
(b) from 70% by weight to 98% by weight, with respect to the total weight of said catalyst, of at least one compound selected from the group consisting of: aluminum oxide (γ-Al₂O₃), aluminum silicate, silicas-aluminas (SiO₂—Al₂O₃), aluminas, zeolites, and metal oxides (such as lanthanum oxide, zirconium oxide, tungsten oxide, thallium oxide, magnesium oxide, zinc oxide, silver oxide);
the sum of (a)+(b) being equal to 100.

4. A process for the production of a diene, comprising the dehydration of at least one alkenol having a number of carbon atoms equal to or greater than 4, in the presence of at least one catalyst comprising coke, wherein said coke, upon analysis by infrared spectroscopy in diffuse reflection (“Diffuse Reflectance Infrared Fourier Transform Spectroscopy”—DRIFT), has at least two peaks at a wavelength comprised between 1450 cm⁻¹ and 1700 cm⁻¹.

5. The process for the production of a diene, according to claim 4, wherein said linear or branched alkenol has the general formula CₙH₂ₙO, n being an integer greater than or equal to 4 and less than or equal to 8.

6. The process for the production of a diene, according to claim 4, wherein said alkenol has a number of carbon atoms equal to 4 and is therefore a butenol; said alkenol is selected from the group consisting of 2-buten-1-ol (crotyl alcohol) (2-Bu-1-OH), 3-buten-2-ol (methyl-vinyl-carbinol) (3-Bu-2-OH), 3-buten-1-ol (allylcarbinol) (3-Bu-1-OH), or mixtures thereof.

7. The process for the production of a diene, according to claim 4, wherein said alkenol having a number of carbon atoms equal to or greater than 4, is obtained directly from biosynthetic processes, or through catalytic dehydration processes of at least one diol.

8. The process for the production of a diene, according to claim 4, wherein when said alkenol has a number of carbon atoms equal to 4, and is therefore a butenol, said butenol is obtained by catalytic dehydration of a butanediol, in the presence of a cerium oxide catalyst, wherein said cerium oxide catalyst is obtained by precipitation, in the presence of at least one base, of at least one cerium-containing compound.

9. The process for the production of a diene, according to claim 7, wherein said diol, derives from the fermentation of sugars; said diol derives from the fermentation of sugars deriving from guayule and/or thistle biomass, including scraps, residues, wastes deriving from guayule and/or thistle, or from their processing.

10. The process for the production of a diene, according to claim 4, wherein said alkenol having a number of carbon atoms equal to or greater than 4 is mixed with a diluent that is selected from the group consisting of: an inert gas, such as nitrogen (N₂), and argon (Ar); or said alkenol is mixed with a compound having a boiling temperature comprised between 25° C. and 150° C. under normal conditions, and a melting temperature less than or equal to 20° C. under normal conditions, selected from the group consisting of water, tetrahydrofuran, cyclohexane, benzene, and mixtures thereof, said diluent is selected from nitrogen (N₂) and water.

11. The process for the production of a diene, according to claim 10, wherein:
if the diluent is selected from inert gases, said process is carried out with a molar ratio of diluent to alkenol (or alkenols) greater than 0.3; and
if the diluent is selected from compounds having a boiling temperature comprised between 25° C. and 150° C. under normal conditions, and a melting temperature less than or equal to 20° C. under normal conditions, said process is carried out with a molar ratio of diluent to alkenol (or alkenols) comprised between 0.01 and 100.

12. The process for the production of a diene, according to claim 4, wherein said process is carried out:
at a temperature comprised between 250° C. and 500° C.; and/or
at a pressure comprised between 5 kPa and 5000 kPa; and/or
in the gas phase or mixed liquid/gas phase; and/or
in a fixed-bed reactor, in a moving-bed reactor, or in a fluidised-bed reactor; and
if it is carried out in a mixed liquid/gas phase, using a continuous stirring reactor (“Continuous flow Stirred-Tank Reactor”, CSTR) containing the catalyst comprising coke in dispersion; and/or
continuously or in batches; and/or
if carried out continuously, the space velocity WHSV (“Weight Hourly Space Velocity”), that is, the ratio of the amount by weight of reactant fed to the reactor to the quantity of catalyst weight in the reactor, is comprised between 0.5 h⁻¹ and 10 h⁻¹; and/or the contact time (τ), calculated as the ratio of the volume of catalyst comprising coke loaded into the dehydration reactor over the volumetric supply flow rate in the reaction conditions, is comprised between 0.01 seconds and 10 seconds.

13. A process for the production of a catalyst comprising coke, wherein said coke, upon analysis by infrared spectroscopy in diffuse reflection has at least two peaks at a wavelength comprised between 1450 cm⁻¹ and 1700 cm⁻¹, said process comprising the dehydration of a mixture of alkenols comprising 3-buten-2-ol (3-Bu-2-OH) and 2-buten-1-ol (2-Bu-1-OH), in the presence of at least one compound selected from the group consisting of: aluminium oxide (γ-Al₂O₃), aluminum silicate, silicas-aluminas (SiO₂—Al₂O₃), aluminas, zeolites, and metal oxides (such as lanthanum oxide, zirconium oxide, tungsten oxide, thallium oxide, magnesium oxide, zinc oxide, silver oxide), said process being carried out at a temperature comprised between 250° C. and 500° C., for a time comprised between 2 hours and 10 hours.
